(19) United States
(12) Patent Application Publication
(10) Pub. No.: US 2010/0125232 A1
(43) Pub. Date: May 20, 2010

(54) Head Suspension Device Vertebral Decompression Method with a Teeth Guard

(76) Inventors: Ivan Fedyaev, London (GB); Elena Fediaeva, London (GB)

Correspondence Address: Ivan Fedyaev, Apartment 53, Galleria Court, Sumner Road, London, SE15 6PW, GB

(21) Appl. No.: 12/534,132

(22) Filed: Aug. 2, 2009

(30) Foreign Application Priority Data
Nov. 14, 2008 (GB) GB0820945.4

(51) Int. Cl. A61F 5/00

(52) U.S. Cl. 602/32

(57) ABSTRACT

Head Suspension Device Vertebral Decompression Method with a Teeth Guard allows a user to raise their legs off the ground in upright vertical position using a teeth guard and grasping a head support frame with both hands. The frame comprises a head back pillow, a chin support and attachment means. After the frame is suspended from an overhead support, the user puts a teeth guard into the mouth to protect the teeth, settles his/her head inside the frame resting the back of their head on the head back pillow and their chin on the chin support, grasps the frame with both hands to protect the neck and gradually raises their legs off the ground thus suspending themselves by head from the frame. In this position the force of the whole body gravitational weight effectively and safely decompresses all the parts of spinal column thus relieving back pain.

This method is intended to relieve and/or to prevent back pain by decompression of human spinal column in upright vertical position using a teeth guard, using a head support frame and raising the user's legs off the ground.

### CROSS REFERENCE TO RELATED APPLICATIONS

This application is a Continuation-in-Part of Application number GB 0820945.4 filed on 14 Nov. 2008 in the United Kingdom by Ivan Fedyaev and entitled: “Head Suspension Device for Vertebral Decompression” in which the same subject matter was disclosed. The Intellectual Property Office Examiner has confirmed that the Device is both “novel and inventive”. Elena Fediaeva was subsequently added to the application as joint applicant and inventor. The only new material added is the information about special grips 10 (see FIGS. 1; 2; 6). The words “ . . . Method with a Teeth Guard” are included in the Title to bring attention to the Method rather than to the Device. Material that describes the inner content of the Method is not new as this Method is the same in both applications (see Application Data Sheet).

### STATEMENT REGARDING FEDERALLY SPONSORED RESEARCH OR DEVELOPMENT

Not Applicable.

### REFERENCE TO A SEQUENCE LISTING, ETC.

Not Applicable.

### BACKGROUND OF THE INVENTION

There are many causes of back pain, natural ageing being one of them. Vertebral column and intervertebral disks bear the entire gravitational weight of our upper body. As years pass the disks become less elastic and thinner until surrounding tissues are compressed and some of their cells damaged. The parts of damaged cells accumulate in the space around the vertebrae. Our body tries to get rid of them by the process of inflammation which leads to inflammatory swelling. The nerves that go out of every vertebra become compressed as well. The result is back pain. This process is often greatly accelerated by aggravating life circumstances. In severe cases a disc may break to form hernia which might manifest itself with sciatica.

One of the ways to relieve chronic back pain is decompression (traction) of spinal (vertebral) column. Vertebral disks are like sponge. They do not have blood vessels of their own and they get nutrition from the vessels surrounding the vertebrae. During traction the space between vertebrae increases, the sponge expands and thus gets more nutrition. It is not yet quite clear why a comparatively short decompressing procedure leads to a fairly long back pain remission. However, decompression does not cure back pain completely and eventually back pain returns. The duration of back pain remission depends on effectiveness of the decompression method and on frequency of the procedure.

Inventors are trying to solve three main problems: effectiveness of decompression method, safety of the device and simplicity of its use. In the early methods different weights were attached to patient's legs with his/her head or shoulders fastened to bed: U.S. Pat. Nos. 4,181,125; 4,257,410; 5,135,537; 6,190,345; 7,033,333. Later special beds (tables) were invented to allow stretching of patient's body with different mechanisms: US 2008 176 721. Generally the stronger the decompressing force used, the more effective decompression is achieved. All these methods using beds and tables may, on some conditions, provide effective decompression but they do not solve the simplicity problem and may be used in hospitals but not at home.

The Method of Inversion was proposed to make a device more effective and simpler to use: U.S. Pat. Nos. 4,410,176; 4,502,682; 5,885,197; 6,464,296; GB 2 444 708. First it was called “Full Body Weight Traction” to highlight that the whole body weight is utilized in the process of decompression and no external weights or mechanisms are attached. Although the claim of the “Full Body Weight” is not correct as the weight of the legs is not utilized in the process of decompression, the Method of Inversion is very effective as it utilizes the most of the body weight. Unfortunately, this Method has a major safety problem as the rush of blood to the head can harm even a young patient.

To solve this safety problem different methods of decompression in upright vertical position were invented. Some of them substituted gravitational weight of the body for the force of patient's muscles (U.S. Pat. Nos. 3,621,839; 1,675,552 A), or for different weights (U.S. Pat. No. 2,658,506 A; 4,869,240; FR 2 686 260), some use different hydraulic, pneumatic or electrical motors (U.S. Pat. Nos. 6,468,240; 6,506,174; 6,971,997).

All these methods are either not effective as they utilize only a part of body weight and deliver only partial decompression in separate parts of vertebral column, or not simple to use, or all of these together. The “Gymnastic Appliance” U.S. Pat. No. 1,675,552 A, which was proposed in 1928 without a teeth guard, in its “could be” modification was not intended to relieve or prevent back pain and the author did not even mention it in his claims.

The most important problem is effectiveness of the method. The Inversion Method is effective because it utilizes most of the body gravitational weight. This weight is the possible strongest (without any mechanism) force applied to all the parts of vertebral column simultaneously. The same should be done in any effective method but it must be done in upright vertical position to exclude the side effect of the rush of blood to the head. It can be done so only if a patient is suspended by head from a head support frame, with his/her legs raised off the ground. In such position the whole body gravitational weight shall be applied to all the parts of vertebral column simultaneously delivering very effective decompression.

At the same time such position shall inevitably create two new problems: considerable strain on the neck and quite strong press on the patient's both jaws. The strain on the neck shall be lessened if the patient grasps the head support frame with both hands to regulate the force of decompression applied to the neck. The strong press on the jaws shall be in addition softened by a teeth guard to protect patient's teeth. Furthermore, the general force of decompression can be easily regulated by the patient gradually bending the knees when their legs are still on the ground. This is especially important when the patient is getting used to the device. What is said in paragraphs [0011] and [0012] in fact is a sketch of the presented Method.

### BRIEF SUMMARY OF THE INVENTION

This Method is based on specific use of our head support frame: a user puts a teeth guard into the mouth, settles his/her head inside the head support frame, grasps the frame with both hands and gradually raises the legs off the ground thus suspending themselves by head from the frame.

Although different head support frames are widely used in many patented methods, the frame presented in our Method differs from them all by having special mobile grips which carry out double function: (1) they allow the patient to firmly grasp the frame with both hands and (2) they fix the chin support of the device during the procedure.

Though these grips may be considered as a “matter of course”, the presented Method still complies with the UK Patents Act 2004 where it is said: “ . . . the “specific use” of a known substance or composition in a method of treatment . . . is treated as new if that specific use was previously unknown”.

If a head support frame can be taken for a “known composition”, then the “specific use” of this previously known composition in our Method is: raising the user's legs off the ground in upright vertical position together with using a teeth guard and together with grasping the head support frame with user's both hands. All the three peculiarities of this specific use are inseparably interconnected: raising the legs off the ground inevitably forces to use a teeth guard as well as to grasp the head support frame with both hands and vice versa, no need in a teeth guard together with no need to grasp the frame means that the user's legs are not raised off the ground which in its turn means that the decompressing force is less than the whole body weight and the decompression is not the most effective.

There is no any patented invention with such a specific use of a head support frame. This specific use of our head support frame is unknown, is new and does not form part of the (previous) state of the art. It forms the new state:
1. Raising the legs off the ground in upright vertical position subjects the user's spinal column to traction with the force of the whole body gravitational weight. It is a very strong force.
2. This force is applied to all the parts of spinal column simultaneously.
3. The same force causes considerable strain on the neck and that neck of the user should be necessarily protected by grasping the head support frame with user's both hands.
4. The same force causes strong press on the user's both jaws and their teeth should be necessarily protected by a teeth guard.

As a result of this new state the decompression has important characteristics it is simultaneously effective, safe and simple to achieve:

1. Effective:
a. because the possible strongest (without any mechanisms) force is used, and
b. because all the parts of spinal column are decompressed at once.

2. Safe:
a. because the user's teeth are protected, because the user's neck is protected,
b. because the force of decompression is easily regulated by the user themselves,
c. because no rush of blood to the head is caused.

3. Simple:
a. because our head support frame is simple, small and light.
b. it is very easy for a person of any age and condition to use it at home.

Patented methods of decompression do not solve the problems of effectiveness, safety and simplicity together. They are either not effective, or not safe or not simple to use or several of these at once.

To overcome all of the above, we propose Head Suspension Device Vertebral Decompression Method with a Teeth Guard allowing a user to raise his/her legs off the ground in upright vertical position together with using a teeth guard and with both their hands grasping the head support frame comprising a frame, a head back pillow mounted on the frame, a mobile chin support firmly connected with special grips at both sides, and attachment means mounted to the frame for suspending the frame from an overhead support. After the head support frame is suspended from the overhead support, the user may put a teeth guard into the mouth to protect the teeth, may settle his/her head inside the head support frame such that they may rest the back of their head on the head back pillow and their chin on the chin support, then the user may grasp the frame on the grips with their both hands to protect the neck and gradually raise their legs off the ground such that they may suspend themselves by head allowing their whole body gravitational weight to decompress their spinal column effectively and safely in all its parts simultaneously.

Advantages of Head Suspension Device Vertebral Decompression Method with a Teeth Guard:

With this Method decompression is performed in upright vertical position which is much more comfortable as compared with Inversion.

With this Method the whole body gravitational weight delivers effective decompression without any mechanisms or external forces which makes it simpler.

This Method makes it possible to decompress the entire vertebral column in all its parts simultaneously thereby not only alleviating back pain in the affected part but also preventing spread of the pathological process to the other parts of the vertebral column.

This Method is safe because the force of decompression can be easily regulated by the user themselves; the user's neck is protected; the user's teeth are protected; and no rush of blood to the head is caused.

This Method is simple because the head support frame is very simple, small and light allowing a patient of any age and condition to often use it at home.

The simplicity of this Method may encourage people who do not yet suffer from back pain to use it for preventative purposes. It is a matter of concern to people in groups of risk: drivers, porters, weight lifters, runners, jumpers and people with a sedentary mode of life.

Note the presented Method in comparison with Inversion Method:
1. Inversion is very effective and simpler than many other methods. Head Suspension Device Vertebral Decompression Method with a Teeth Guard has positive characteristics of the Inversion Method: see [0022] and [0023]. The presented method is even more effective because the weight of the legs is also utilized in the process of decompression.
2. Head Suspension Device Vertebral Decompression Method with a Teeth Guard does not have negative characteristics of the Inversion Method, thus it has [0021], [0024] and [0025].
3. In connection with [0026] it should be pointed out that Inversion Method is useful for preventative purposes as well though some important contraindications restrict its use. Inverted position causes a rush of blood to the head which can result in hemorrhage. Accordingly, Inversion must not be used by people taking anti-coagulants or suffering from glaucoma, retinal detachment, high blood pressure, a heart illness, an ear disease and from some other ailments and disorders. Any of the illnesses mentioned can be in so called latent period when a person is not aware of the illness. To use Inversion to prevent back pain at this time can strongly accelerate pathological process.

### BRIEF DESCRIPTION OF THE DRAWINGS

FIG. 1 represents a schematic top view of the portable head support frame comprising a frame 1, a head back pillow 2, a chin support 3 connected with special grips 10, and attaching means 4 with buckles 9.

FIG. 2 represents a schematic longitudinal section of the chin support 3. Together with other figures it helps understand function of the grips 10.

FIG. 3 shows a usual double mouth guard 5 which we recommend to use as a teeth guard. A double mouth guard is available at sport shops though a teeth guard can be made of any sufficiently elastic and chemically not harmful material.

FIG. 4 shows the end of the first stage of decompression procedure in a schematic perspective view. A portable model (see FIG. 1) of Head Suspension Device is being used.

FIG. 5 shows a schematic perspective view of the final stage of decompression procedure with the portable model. This figure is desirable to be included on the front page of the patent application publication to illustrate the invention.

FIG. 6 represents a stationary model of Head Suspension Device in which the head support frame 1 is included with the help of bolts 6 in metallic cage 13. With using additional attaching means 12 this model is more stable. At the same time it is not easy to attach and may be recommended only if there is a constant place for the device. This FIGURE proves that the Head Suspension Device can be made of different forms and materials.

FIG. 7 shows, as well as FIG. 4, the end of the first stage of decompression procedure. A stationary model of Head Suspension Device is being used.

### DETAILED DESCRIPTION OF THE INVENTION

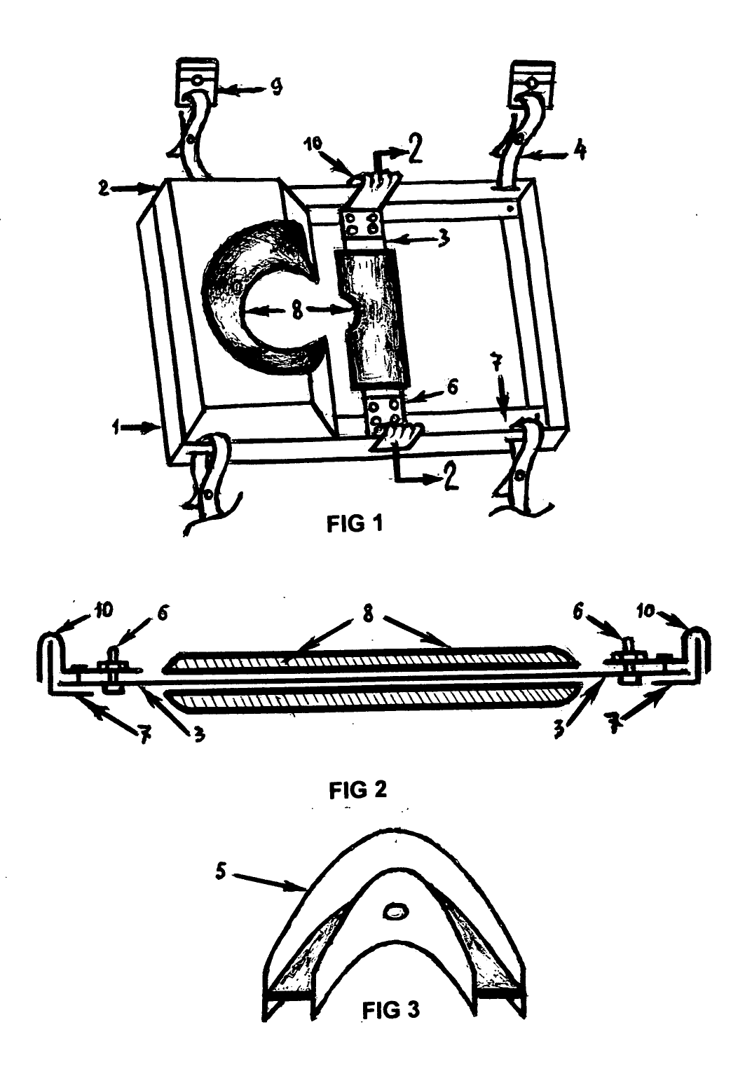

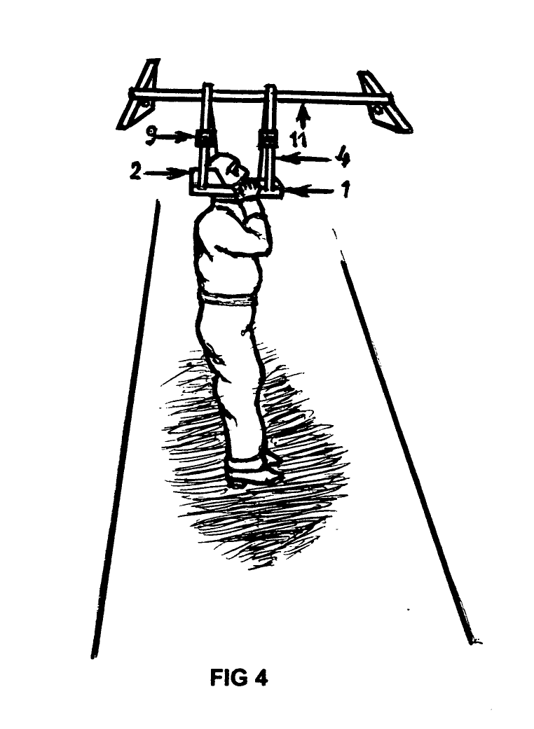

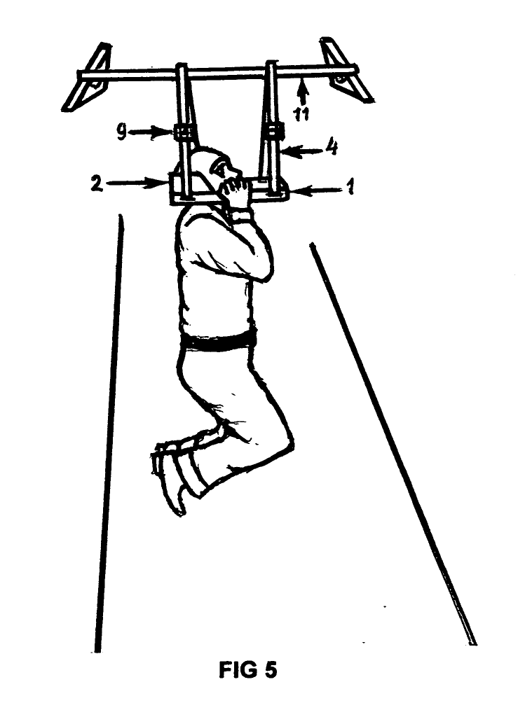

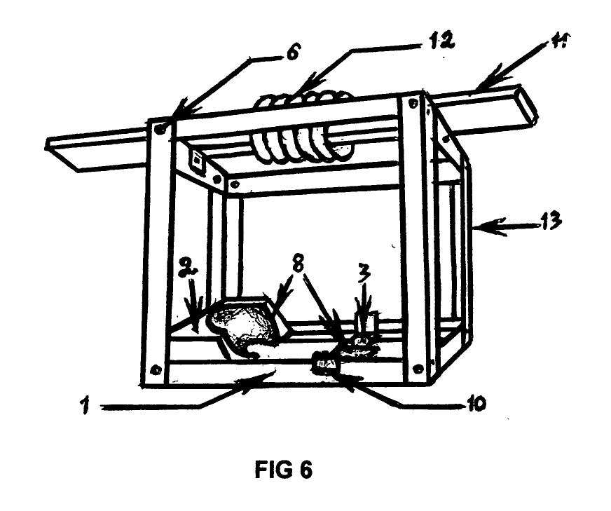

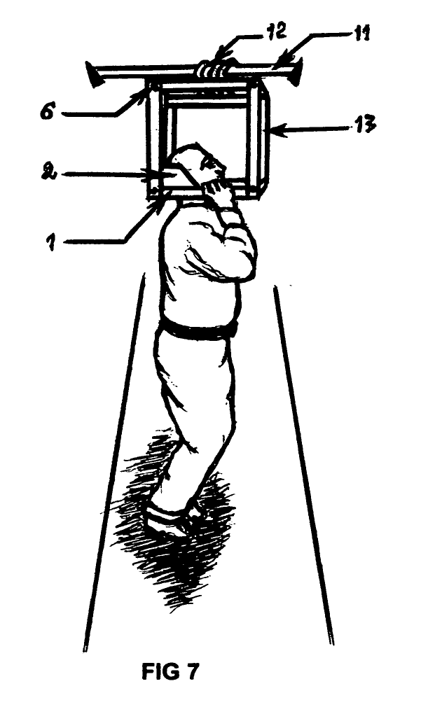

Referring to FIGS. 1-7, during the first stage of decompression procedure the user suspends the head support frame from an overhead support with the help of attaching means 4 and 11 or 12. Attaching means 4 can be made of different materials and have usual buckles 9 to change the length. After the frame is suspended from the overhead support, the user puts a teeth guard (preferably a double mouth guard 5) into the mouth to protect the teeth. Then he/she settles their head inside the frame 1 such that they rest the back of their head on the head back pillow 2 and their chin on the chin support 3 moving the latter on sleigh runners 7. To make it more comfortable for the user, the head back pillow 2 and the chin support 3 are covered with soft material 8. The user then grasps the grips 10 with both their hands to regulate the decompressing force and thus to protect their neck. The grips 10 are movable as they are connected by bolts 6 with the chin support 3 which can move on the sleigh runners 7. These grips carry out double function: they serve (1) to firmly grasp the head support frame with user's both hands to protect their neck and (2) to exclude uncontrollable moving of the chin support 3 along the sleigh runners 7 during the procedure. At the end of the first stage of decompression procedure the user begins to gradually bend their knees (see FIGS. 4 and 7).

The final stage of decompression procedure is shown on FIG. 5. It is a short but very important part of the whole procedure. In fact, it is the aim of the Method. In this stage the user's legs are raised off the ground with the whole body suspended by head from the head support frame. This position subjects all the parts of spinal column to traction with the force of the whole body gravitational weight.

Different parts of spinal column are differently sensitive to this force. The most sensitive are the vertebrae of the neck. The user feels great strain on the neck and has to grasp the head support frame with both hands partially to lift the body weight. This lifting lessens the force applied to the neck but the lessened force is always strong enough for effective decompression to relieve and prevent neck pain and all this is regulated by the user themselves. The decompressing force applied to pectoral and lumbar parts of vertebral column is not lessened by grasping the head support frame with the user's both hands.

Other sensitive areas are user's upper and lower jaws. The strong press on the jaws is reduced simultaneously with lessening the force applied to the neck by grasping the head support frame with user's both hands. To protect the teeth, the decompressing force is additionally softened by a teeth guard (a double mouth guard 5 should be preferably used).

Due to these sensitive areas of human body and a very strong decompressing force, the final stage of decompression procedure is short. Even with a teeth guard and grasping the head support frame with both hands a trained user can extend the final stage, when the legs are raised off the ground, to not more than 5 minutes. Without a teeth guard and without grasping the head support frame with both hands one would be able to endure that strong force only for several seconds and those seconds would not be safe.

The shortness of the final stage of decompression procedure is successfully compensated by the effectiveness, safety and simplicity of the Method: a person of any age and condition can use it at home regularly every day. Regular use of the decompression procedure makes back pain remissions longer and longer which helps return to normal mode of life.

The final stage of decompression procedure shown on FIG. 5 where the user's legs are raised off the ground is important. However, any patient, especially weak, sick or old, can benefit from using this Method even without raising their legs off the ground. A patient can instead lengthen the time of the procedure and perform careful circular movements like hula hooping to alleviate pain. Later on they can gradually increase the force of decompression and even attempt to raise their legs off the ground to make decompression the most effective.

1. Head Suspension Device Vertebral Decompression Method with a Teeth Guard allowing a user to raise his/her legs off the ground in upright vertical position together with using a teeth guard and with both hands grasping the head support frame comprising a frame, a head back pillow mounted on the frame, a mobile chin support and attachment means mounted to the frame for suspending the frame from an overhead support; wherein, in use with the head support frame suspended from the overhead support
the user may put a teeth guard into the mouth to protect the teeth,
may settle his/her head inside the head support frame such that they may rest the back of their head on the head back pillow and their chin on the chin support,
grasp the frame with their both hands to protect the neck and
gradually raise their legs off the ground such that they may suspend themselves by head allowing their whole body gravitational weight to decompress their spinal column effectively and safely in all its parts simultaneously.

2. Special grips allowing the user to firmly grasp the frame with user's both hands and to exclude uncontrollable moving of the chin support during the decompression procedure.
